(19) United States
(12) Patent Application Publication
(10) Pub. No.: US 2013/0123662 A1
(43) Pub. Date: May 16, 2013

(54) JOINT BIOPSY NEEDLE FOR TAKING TISSUE SAMPLES

(71) Applicant: Markus Hipp, Kolbingen (DE)

(72) Inventor: Markus Hipp, Kolbingen (DE)

(73) Assignee: Hipp Medical AG, Kolbingen (DE)

(21) Appl. No.: 13/701,096

(22) PCT Filed: May 27, 2011
(86) PCT No.: PCT/EP11/02639

§ 371 Date: Nov. 30, 2012

(30) Foreign Application Priority Data
Jun. 1, 2010 (DE) 10 2010 017 185.9

(51) Int. Cl. A61B 10/02

(52) U.S. Cl. CPC A61B 10/0233
USPC 600/564

(57) ABSTRACT

The invention relates to a joint biopsy needle (1) for taking tissue samples and to a method therefor. The joint biopsy needle (1) has a trocar tip (2) and a substantially rigid guide sleeve (11), which adjoins a base surface (23) of the trocar tip (2) and has a lateral opening (12) in a region adjacent to the trocar tip (2). The biopsy needle (1) also has a plunger (3), which can be displaced in the guide sleeve (11) in the longitudinal direction thereof and has a cutting blade (31) on a side (30) that faces the trocar tip (2), wherein the tissue which is to be taken in the region of the lateral opening (12) of the guide sleeve (11) and can enter the guide sleeve (11) through the lateral opening (12) can be severed by the cutting blade (31). The guide sleeve (11) is inserted into a handle (6). To this end, the guide sleeve (11) is curved at least in one location between the trocar tip (2) and the handle (6), wherein the displaceable plunger (3) can be displaced by means of a flexible force transmitting element (8) that is guided in the guide sleeve (11). Thus a biopsy needle is obtained which is simple to operate and by means of which tissue samples can be taken effectively even in sampling locations that are inconveniently located and at times difficult to access.

The present invention relates to a joint biopsy needle for taking tissue samples in accordance with the preamble of claim 1 and a method herefor in accordance with the preamble of claim 20.

Such joint biopsy needles customarily comprise a trocar tip and a guide sleeve which adjoins a base surface of the trocar tip and has a lateral opening in a region adjacent to the trocar tip. Moreover such biopsy needles frequently comprise a plunger which can be displaced in the guide sleeve in the longitudinal direction thereof and has a cutting blade on a side that faces the trocar tip, wherein tissue which is to be taken in the region of the lateral opening of the guide sleeve and can enter the guide sleeve through the lateral opening can be severed by the cutting blade. The guide sleeve is immobilized in a handle.

In cases of rheumatism patients, for example, inflammations of a joint occur on the synovial membrane. This often results in swelling and in the spread of bacteria into surrounding tissue. In order to administer suitable medication in such cases it is necessary to ascertain the type of bacterium in the tissue.

In order to realize this, it is necessary to take a tissue sample that can be examined in a laboratory later on. Conventional tissue taking devices known from the prior art are, e.g., hollow biopsy needles whereby it is possible to take tissue samples of human, animal, plant and/or technical structures by using various mechanisms.

In the framework of such tissue sampling, lateral and coaxial cutting mechanisms in combination with screw mechanisms or mechanisms operating according to the corkscrew principle are utilized, for example, particularly in the case of the so-called needle biopsy. Vacuum-assisted biopsy is moreover also employed by making use of a vacuum-generating syringe/piston mechanism or an external vacuum apparatus, wherein tissue to be taken is sucked into the biopsy needle from the tissue compound to be examined by the negative pressure generated in the biopsy needle, for instance through a lateral window of a proximal hollow needle area. The tissue sucked into the hollow needle area may then be torn off by the negative pressure or severed by a cutting implement.

For example, International Patent Application PCT/DE03/00844, published as WO 03/077767 A1 and discussing a vacuum biopsy device, discloses a biopsy device for taking tissue samples which consists of a hand piece into which a hollow biopsy needle is inserted, wherein a portion of the rectilinear biopsy needle protruding from the hand piece is introduced with its sampling chamber into the tissue being investigated, and the tissue is sucked into the sampling chamber by vacuum and then severed by means of a sample separating mechanism and finally removed.

These devices and methods are thus well-suited for taking a sample from easily accessible tissue. If, however, a sample taking region is concealed by some other body part and/or tissue region that must not be injured or touched, it is possible only with difficulty or not at all to take a corresponding tissue sample with the aid of the above-mentioned devices.

Accordingly, it is the object of the present invention to provide a joint biopsy needle for taking tissue samples that is easy to operate even in awkward sampling locations, as well as a method herefor.

In accordance with the invention this object is achieved through a joint biopsy needle having the features of claim 1. The object is furthermore achieved through the method including the method steps according to claim 20.

In accordance with the invention a joint biopsy needle for taking tissue samples is provided, comprising a trocar tip and a substantially rigid guide sleeve which adjoins a base surface of the trocar tip and which has a lateral opening in a region adjacent to the trocar tip, wherein the biopsy needle has a plunger which can be displaced in the guide sleeve in the longitudinal direction thereof and has a cutting blade on a side that faces the trocar tip, wherein the tissue which is to be taken in the region of the lateral opening of the guide sleeve and can enter the guide sleeve through the lateral opening can be severed by the cutting blade.

The guide sleeve is inserted into a handle, with the guide sleeve in accordance with the invention being for the first time curved at least in one location between the trocar tip and the handle, wherein the plunger can be displaced by means of a flexible force transmitting element that is guided in the guide sleeve.

What is of particular advantage here is the small and simple construction and operation of the joint biopsy needle which is nevertheless extremely effective owing to the curved guide sleeve.

The curvature of the guide sleeve makes it easier for the person carrying out the treatment to position the biopsy needle in a desired tissue taking region of the body. In this case the handle is not a hindrance even in the event of oblique introduction inasmuch as it does not come to rest on the body, which would otherwise result in a restricted freedom of movement.

Moreover the curvature of the guide sleeve makes tissue regions accessible which could not be reached at all or only with great difficulty with a straight configuration of the guide sleeve or of the biopsy needle, respectively, for example when penetrating with the biopsy needle into a tissue region that is concealed by a body part which must not be injured or touched.

The flexible force transmitting element is capable of following any configuration of the guide sleeve and thus of transmitting at all times the required force to the cutting plunger in order to correspondingly move the latter inside the guide sleeve. As the person carrying out the treatment requires neither additional equipment elements nor an otherwise necessary power source, tissue taking may be prepared and executed quickly, and easily.

Owing to the simple design of the component parts of the biopsy needle of the invention, the latter is a more cost-efficient solution when compared to vacuum biopsy needles and/or biopsy needles with additional electric equipment.

Advantageous developments of the joint biopsy needle of the invention are subject matter of appended claims 2 to 19.

Thus, the flexible force transmitting element may, e.g., be realized in the form of a plastic tube which may be provided quickly and easily and thus constitutes a cost-efficient solution. Such a hollow body may moreover serve as a delivery passage for a medicament to be injected into the region under examination.

On its inside the plunger may have a hollow space which is open toward the cutting blade and in which the tissue to be collected may be received. Inside this hollow space a taken tissue sample may be received for as long until the biopsy needle is extracted from the body, to then be removed from there. The hollow space may be encapsulated by form-fit with a base surface of the trocar tip such that tissue contaminated, in a given case, by bacteria or other impurities will not get into contact with other tissue regions or body parts when the biopsy needle is extracted from the body.

A trocar tip may moreover be realized in the form of a cutting tip having a plurality of sides with concave cutting blades, so that a cutting force may be transmitted better in a linear distal direction and penetration to the region to be examined is facilitated.

Moreover the trocar tip may on at least one side have at least one bore whereby a body fluid is allowed to flow through the trocar tip. The risk of certain fluid circuits of the body being blocked by the trocar tip during the examination may thus be restricted to a minimum.

On the end of the joint biopsy needle opposite the trocar tip, at the distal end of the handle, a coupling means for a pressure generating device may be provided, by means of which a drug may be injected into the region to be treated via a continuous cannulation through the handle, the force transmitting element, the plunger, and/or the trocar tip while the biopsy needle is located in the region under examination. As a result the biopsy needle can serve not only as a tissue sample taking device but also as a drug injection means. A precautionary or subsequent second puncture into the body in order to introduce the drug may thus be avoided.

Moreover it may be possible to transmit a force to the flexible force transmitting element via an actuating device and a force transmitting piston which is guided in the handle, wherein a first reception is located at an end of the plunger facing the force transmitting piston, and a second reception for the force transmitting element is located at an end of the force transmitting piston facing the plunger. The actuation element enables the person carrying out the treatment to operate the biopsy needle easily and quickly without any complex additional equipment. In addition the assembly of force transmitting piston, plunger, and force transmitting element via the first and second receptions allows simple and rapid assembling and disassembling.

Moreover a distal shaft of the force transmitting piston and an associated guide hole in the handle wherein the force transmitting piston is guided, may for the first time surprisingly be formed so as to be secured against rotation, in particular in a respective eccentric and complementary manner. As a result the shaft can not be rotated any more relative to the handle so that the radial position will always remain the same, and the force transmitting element may be prevented from being disconnected from the first and second receptions as the result of a torsional force.

Inside the handle a reset spring member for the plunger may moreover be arranged which, following completion of the cutting operation, returns the cutting plunger into its basic position via the force transmitting element and the force transmitting piston. Thus the biopsy needle may easily be operated with one hand. The reset spring member is preferably accommodated in the handle such as to be prevented from getting into contact either from the inside or outside with tissue or other substances, whereby a particularly prolonged and reliable operation may be ensured.

In accordance with another aspect of the present invention according to claim 20 a method for taking tissue samples is being shown. In this method the joint biopsy needle of the invention is at first introduced into a region to be examined in a body. Subsequently, once the biopsy needle has been introduced into the desired position, the plunger is actuated via the actuating device, the force transmitting piston, and the force transmitting element so as to sever tissue to be collected in the region of the lateral opening of the guide sleeve, which could enter the guide sleeve through the lateral opening, by means of the cutting blade and receive it in the hollow space of the plunger. Then, when the tissue to be taken is inside the hollow space, the joint biopsy needle is extracted from the region under examination. Finally, after the biopsy needle has been extracted from the body, the tissue sample may be removed from the hollow space. This brings about the same advantages as already pointed out in the foregoing in connection with the biopsy needle.

In the following the invention shall be described in more detail through the embodiments represented in the annexed figures, wherein:

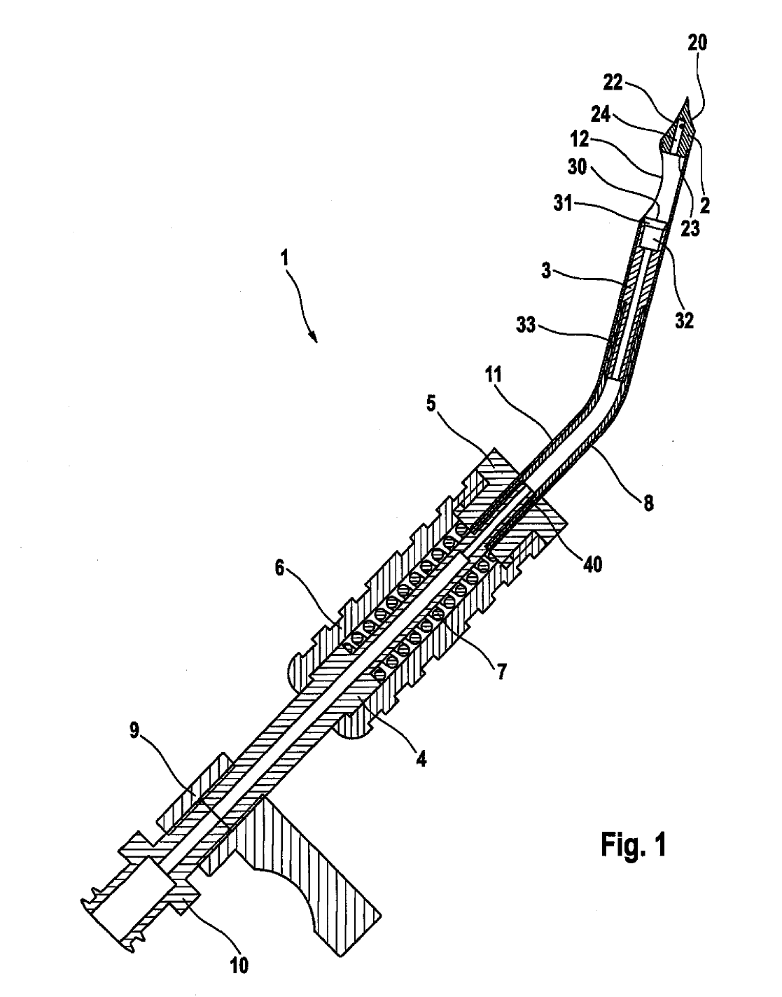

FIG. 1 is a sectional view of a joint biopsy needle or biopsy needle in accordance with the invention;

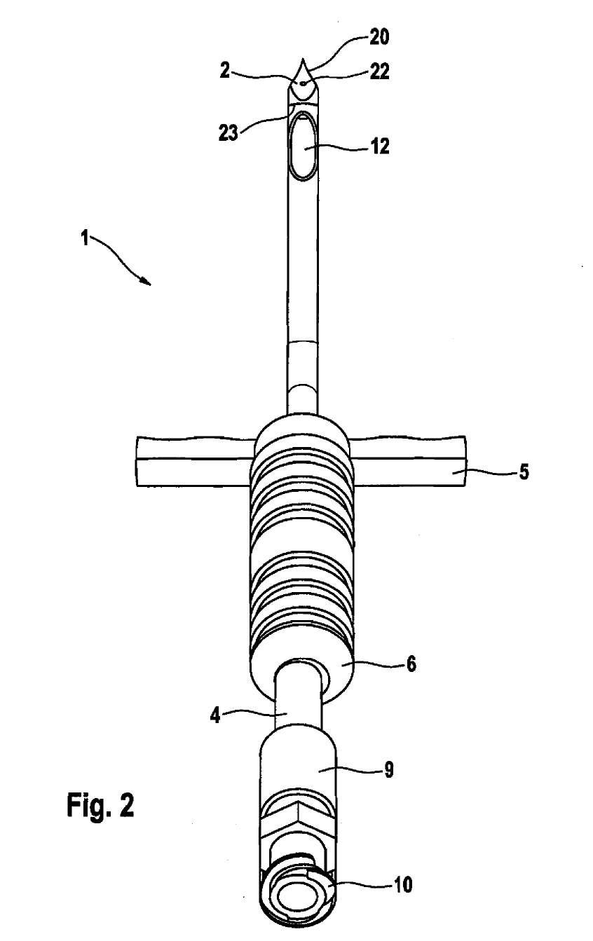

FIG. 2 is a lateral view of the biopsy needle in accordance with the invention;

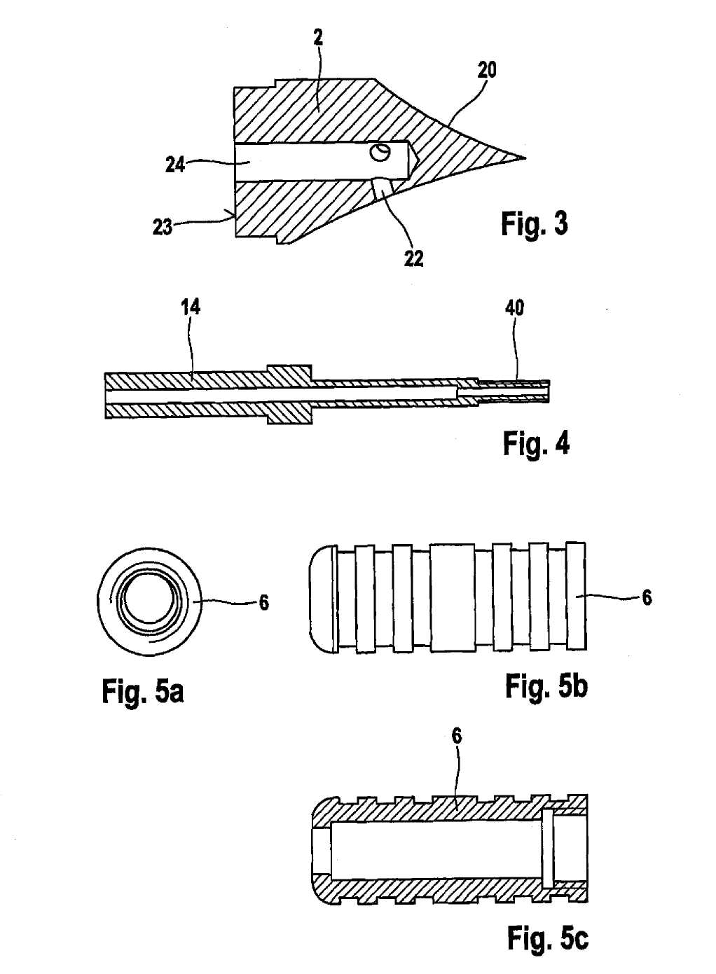

FIG. 3 is a sectional view of a trocar tip of the biopsy needle in accordance with the invention;

FIG. 4 is a sectional view of a force transmitting plunger of the biopsy needle in accordance with the invention;

FIG. 5a is a top view of a handle of the biopsy needle in accordance with the invention;

FIG. 5b is a lateral view of a handle of the biopsy needle in accordance with the invention;

FIG. 5c is a sectional view of a handle of the biopsy needle in accordance with the invention;

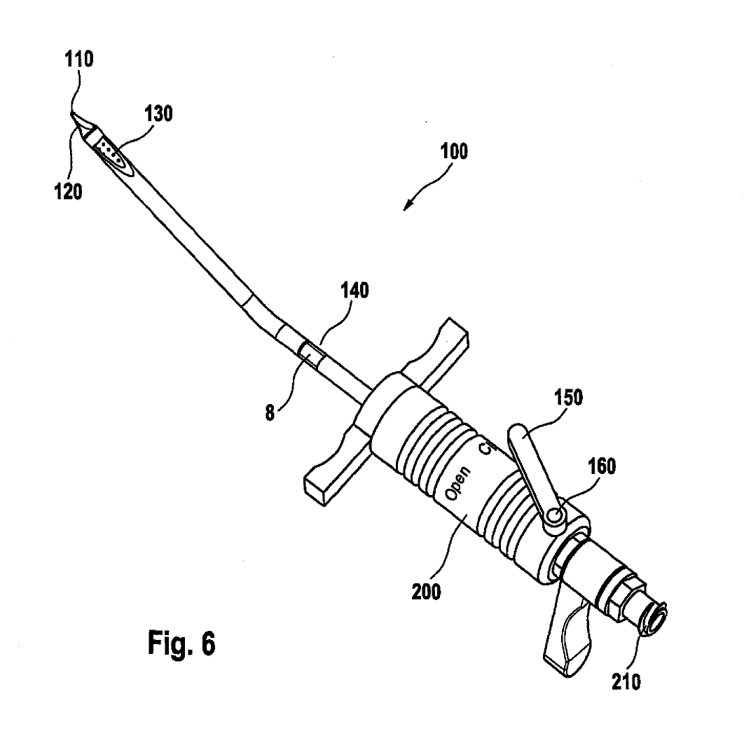

FIG. 6 is a perspective view of an alternative embodiment of the biopsy needle;

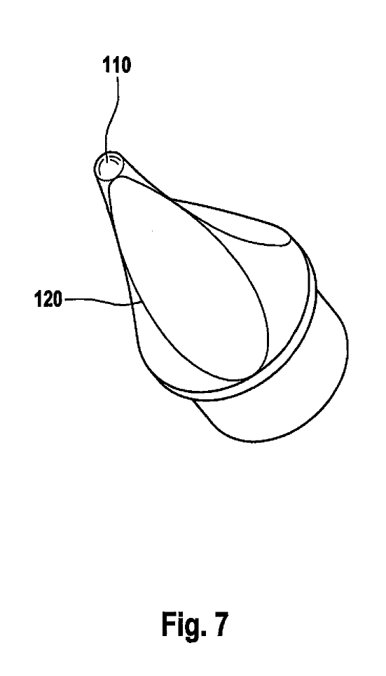

FIG. 7 is an enlarged view of the trocar tip according to the alternative embodiment of the biopsy needle;

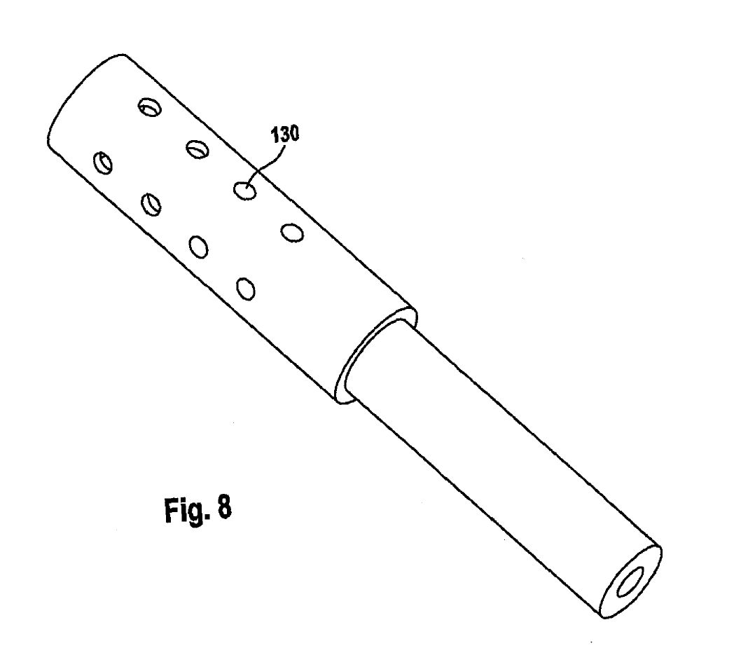

FIG. 8 is an enlarged view of the cutting plunger according to the alternative embodiment of the biopsy needle;

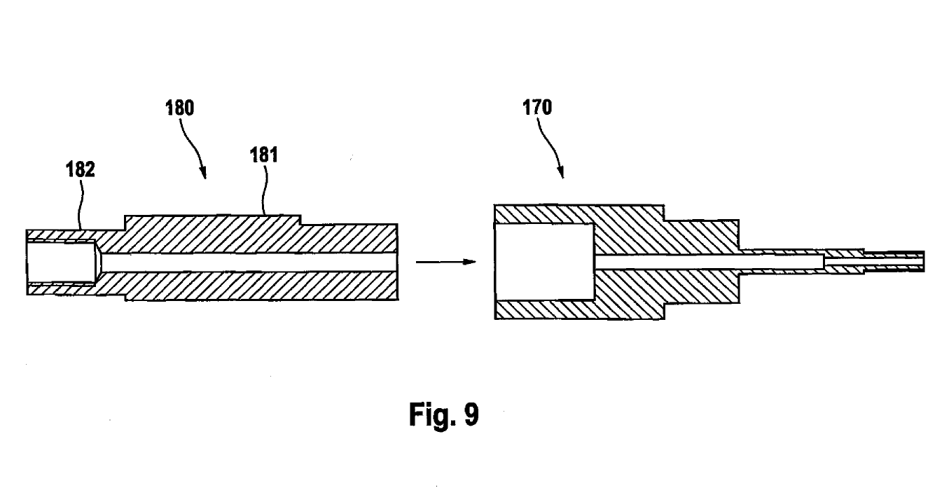

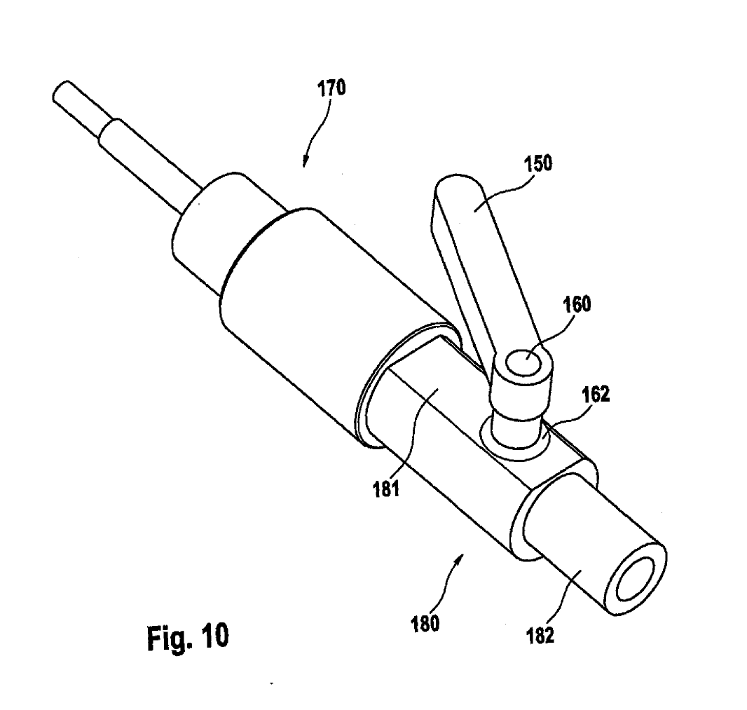

FIG. 9 is a sectional view of a split force transmitting piston according to the alternative embodiment of the biopsy needle;

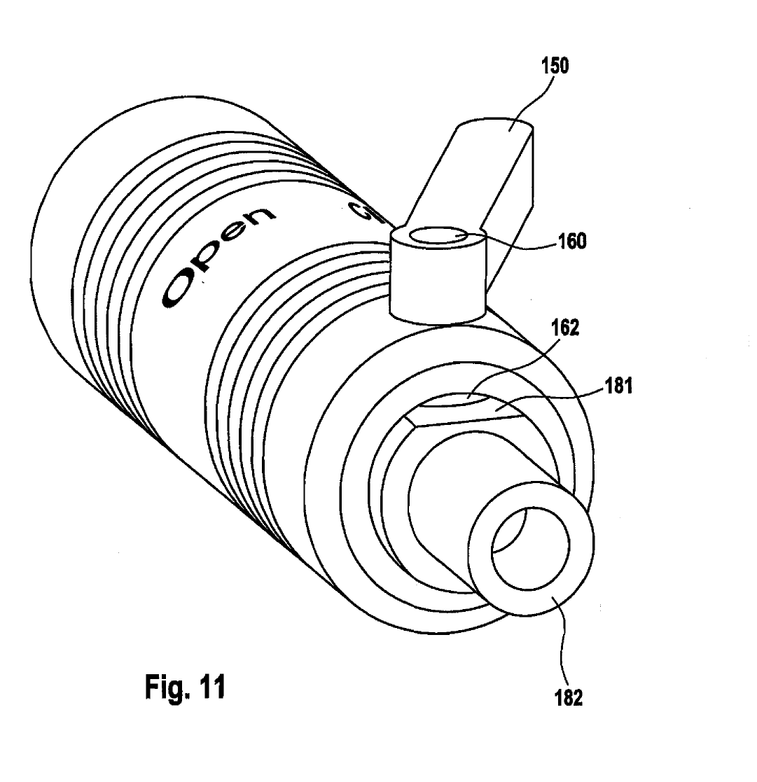

FIG. 10 is a perspective view of the force transmitting piston including a clamping member with lever in accordance with the alternative embodiment of the biopsy needle without a handle;

FIG. 11 is a perspective view of the force transmitting piston including a clamping member with lever in accordance with the alternative embodiment of the biopsy needle with a handle;

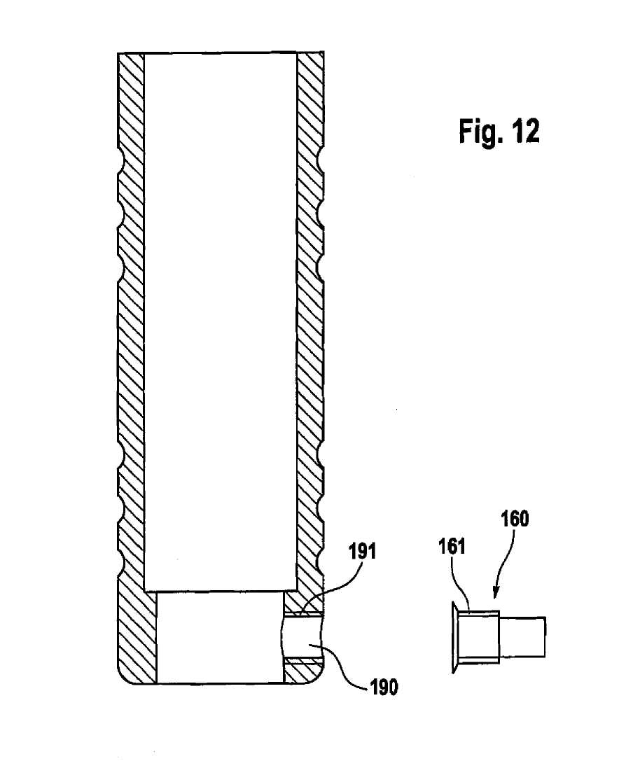

FIG. 12 is a sectional view of the handle according to the alternative embodiment of the biopsy needle with the clamping member according to the alternative embodiment of the biopsy needle; and

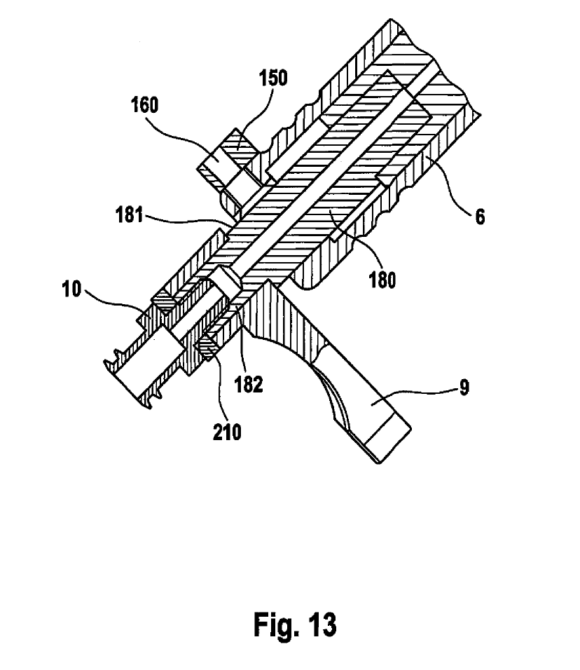

FIG. 13 is a sectional view of the rear part of the alternative embodiment of the biopsy needle.

FIG. 1 and FIG. 2 show a biopsy needle 1, with a sectional view of the biopsy needle 1 being shown in FIG. 1. A trocar tip 2 of the biopsy needle 1 is here realized in the form of a cutting tip having a plurality of sides with concave cutting blades 20.

As is represented enlarged in FIG. 3, the trocar tip 2 is realized with a three-sided cutting tip and has a bore 22 on each one of the three sides, with these bores 22 meeting centrally in the trocar tip 2. The trocar tip 2 moreover comprises a passage 24 between the intersection point of the bores 22 and a base surface 23 of the trocar tip 2.

FIG. 1 further shows a guide sleeve 8 which adjoins the base surface 23 of the trocar tip 2 and which has a lateral opening 12—presently having an oval shape—in a front region near the trocar tip 2. The guide sleeve 8 is made of a substantially rigid, biocompatible material.

As represented in FIG. 1, the guide sleeve 11 presents a curvature in a location between the trocar tip 2 and a handle 6. Angle and radius of the curvature may be realized at will, depending on the wishes and needs of the person carrying out the treatment or the requirements of the respective treatment. In the case of the present embodiment the angle of the curvature is at about 30 degrees.

The biopsy needle 1 moreover contains a plunger 3 which is displaceable in the guide sleeve 11 in the longitudinal direction thereof and which has on a front side 30 facing toward the trocar tip 2a cutting blade 31, wherein the tissue to be taken in the region of the lateral opening 12 of the guide sleeve 11 and which may enter the guide sleeve through the lateral opening can be severed by the cutting blade 31. The cutting blade 31 is formed over the entire circumference of the front side 30. The cross-section of the plunger 3 is adapted to the shape and the configuration of the guide sleeve.

In addition the plunger 3 comprises on its inside a hollow space 32 which is open toward the cutting blade 31 and in which the tissue to be collected may be received. Shape and volume of the opening may be configured according to wish, with a cylindrical bore having been found to be particularly proficient in utilization and manufacture in the present preferred embodiment.

As represented in FIG. 1, the biopsy needle 1 moreover comprises a flexible force transmitting element 8 which is guided in the guide sleeve 11 and whereby the plunger 3 may be displaced in the guide sleeve 11, with the force transmitting element 8 being realized of a flexible plastic tube in the present embodiment.

The guide sleeve 11 is here inserted in a handle 6. The force acting on the force transmitting element 8 can be transmitted via an actuating device 9 and a force transmitting piston 4 which is guided in the handle 6, with a first reception 33 and a second reception 40 for the force transmitting element 8 being located on an end of the plunger 3 facing the force transmitting piston 4 and on an end of the force transmitting piston 4 facing the plunger 3, respectively.

The first reception 33 and the second reception 40 are preferably realized as a thread on which the force transmitting element 8 described in the foregoing may be screwed.

As is shown in FIGS. 4, 5a, 5b, and 5c, a distal shaft 14 of the force transmitting piston 4 and an associated guide hole in the handle 6 each have an eccentric formation. The distal shaft 14 of the force transmitting piston 4 can thus not be rotated any more relative to the associated guide hole in the handle 6, so that the radial position will always remain the same. As a result, in the case of the present screw connection having the first and second receptions 33, 40, the force transmitting element 8 or the plastic tube can not be opened any more by twisting in the wrong direction.

In the biopsy needle or in the handle, respectively, there is moreover arranged a reset spring member 7 for the plunger 3 which presently has the form of a helical spring and which, following completion of the cutting operation, returns the plunger 3 into its basic position via the force transmitting element 8 and the force transmitting piston 4. In the present embodiment the pre-stress is applied to the reset spring member 7 via a retaining member 5 located on the proximal end of the handle 6 and the force transmitting piston 4 formed on the distal end of the handle.

On the end of the biopsy needle 1 opposite the trocar tip 2, on the distal end of the handle 6, there is furthermore provided a coupling means 10 for a pressure generating device (not represented), by means of which a drug may be injected into the region to be treated via a continuous cannulation through the handle 6, the tube-type force transmitting element 8, the plunger 3, and the trocar tip 2 while the biopsy needle 1 is located in the region under examination. The coupling means 10 is presently realized in the form of a Luer-Lock connection on which a syringe (not represented) may be attached.

In the following a method for taking tissue samples shall be described in detail. In the process, the biopsy needle 1 of the invention is at first introduced into a region to be examined in a body, with the trocar tip 2 cutting a path through the body as far as the tissue taking region.

The person carrying out the treatment grasps the biopsy needle in such a way as to place the middle finger and index finger on the retaining member 5 and the thumb on the actuating device 9. While penetration into the body is taking place, the actuating device 9 may or may not be kept depressed according to the judgment of the person carrying out the treatment.

Once the biopsy needle 1 has been introduced to the desired position, the plunger 3 is depressed via the actuating device 9, the force transmitting piston 4, and the force transmitting element 8 so as to sever tissue to be collected, which could enter into the guide sleeve 11 through the lateral opening 12, in the region of the lateral opening 12 of the guide sleeve 11 with the aid of the cutting blade 31 and receive it in the hollow space 32 of the plunger 3.

Here the force brought to bear on the force transmitting piston 4 via the actuating device 9 is transmitted to the force transmitting element 8 which transmits the resulting force to the plunger 3 while being limited by the wall of the guide sleeve 11. The force transmitting element 8 can not buckle in an inward direction because the transverse contraction will always act on the wall of the guide sleeve 11. The force transmitting element 8 thus transmits the force along the desired arc or the provided curvature(s) of the guide sleeve 11.

Then, when the tissue to be taken is located in the hollow space 32, the biopsy needle is removed from the region under examination while the plunger 3 rests in form-fit against the base surface 23 of the trocar tip, i.e., while the actuating device 9 is fully depressed.

Finally, after the biopsy needle has been extracted from the body, the actuating device 9 may be released. Now, after the plunger 3 has assumed its basic position again, the tissue sample may be removed from the hollow space 32.

While the biopsy needle 1 is located in the tissue taking region or some other region inside the body, a medicament, for instance a drug for the treatment of the region under examination, may be injected into the body through the biopsy needle 1. To this end a syringe/piston device whereby the medicament or drug is injected through the force transmitting piston 4, the force transmitting element 8, the plunger 3, and the trocar tip 2 into the selected region in the body, may be attached to the coupling means 10. This may be done either through the bore 22 (passage 24 and bores 22) with the plunger 3 depressed, or through the lateral opening 12 when the plunger 3 is not depressed.

Besides the discussed embodiment, the invention allows for further design approaches.

As is represented in FIGS. 1 and 5a to 5c, the handle 6 is provided with a structured surface which may be realized according to wish in further embodiments. Even smooth and/or slip-preventing surfaces are equally conceivable.

Instead of the three-sided trocar tip 2 the latter may present any other number and shape of cutting surfaces or edges, and may be realized in the form of a conical, concave, or convex round tip. The trocar tip 2 may then have at least one bore 22 on at least one side face 20. Alternatively even a single continuous bore or randomly shaped opening, or a number of bores or openings through the trocar tip 2 corresponding to the number of cutting surfaces may be formed so as to also meet in a central position.

In the preferred embodiment the curvature of the guide sleeve 11 is executed at an angle of about 30 degrees. For further applications it may, however, also be situated in a range between 20 degrees and 60 degrees, and in a particularly preferred embodiment of the invention in a range between 25 degrees and 35 degrees. Further, additional curvatures of the guide sleeve 11 at other angles and/or radii are equally conceivable.

Instead of the substantially rigid guide sleeve 11 a guide sleeve of a flexible biocompatible material is also conceivable.

In general, however, the substantially rigid guide sleeve 11 is preferred. The rigid guide sleeve may be made, e.g., of hardened steel 1.4034 (hardened to 52 to 54 HRC) or of hardened steel 1.4057 (hardened to 52 to 54 HRC).

As a result of a stiff or rigid guide sleeve, secure and accurate penetration into the target region may be ensured despite the bending, thrust and/or torsional stresses occurring during a treatment. In general so-called knife steels, INOX steels or VA steels may also serve as a starting material for the guide sleeve, wherein it is an aim to provide an optimum of good biocompatibility, good chemical resistance, non-rusting properties, high hardness, and martensitic texture.

Such steels which are suited for a substantially stiff or rigid guide sleeve have, for example, a tensile strength of 780 MPa to 1,000 MPa, preferably 800 MPa to 980 MPa, in a particularly preferred manner 830 MPa to 950 MPa. Suitable steels present, e.g., an elongation of 10% to 14%, preferably about 12%. The modulus of elasticity of suitable steels is situated, e.g., in a range of 205 N/mm² to 225 N/mm², preferably about 215 N/mm².

As regards the apparent limit of elasticity of suitable steels, it is possible to specify an example range of 550 to 700 N/mm², preferably of 575 to 625 N/mm², in a particularly preferred manner 600 to 650 N/mm².

In the framework of example material tests, i. a. for examining the rigidity of the guide sleeve, the following two steels were found to be particularly well-suited examples:

1.4034:
Flexural strength /tensile strength: 850 MPa

### Elongation: 12%

Modulus of elasticity: 215 N/mm²×10³
Limit of elasticity: 600 N/mm²
1.4057:
Flexural strength/tensile strength: 930 MPa

### Elongation: 12%

Modulus of elasticity: 215 N/mm²×10³
Limit of elasticity: 650 N/mm²

These parameters, named by way of example in the foregoing, may be determined, e.g., by the tests known from mechanical material testing. By way of example, reference should be made to the most usual, classical tensile test which is described in detail in DIN EN 10002 (formerly DIN 50125, DIN 50145).

In the joint biopsy needle in accordance with the invention being discussed, the material presently proposed by way of example for a rigid guiding aid may i. a. also be employed particularly well for the trocar tip and/or the plunger.

The guide sleeve 8 is not restricted to a circular cross-section but may also be formed with an elliptic, rectangular, or any other cross-sectional shape.

The cutting blade of the plunger 3 may in an alternative embodiment be formed over only a part of the front side 30 or the border thereof and/or in the inner region of the front side 30 of the plunger 3.

In a further embodiment the tube-type, flexible force transmitting element 8 may be realized, e.g., of an elliptic or polygonal hollow or solid profile. Moreover it may also be plug-connected, hooked, bonded, soldered and/or welded with the plunger 3 and the force transmitting piston 4, depending on the embodiment of the force transmitting element 8 and the plunger 3.

If there is no curvature or only a slight curvature of the guide sleeve 11, or the force transmitting element 8 is not screw-connected but—as mentioned in the foregoing—connected to the plunger 3 and the force transmitting piston 4 in some other manner, it is possible to do away with the eccentric formation of the rear shaft 14 of the force transmitting piston 4 and the bore in the handle 6, or the latter may have a centric formation.

Instead of the reset spring member 7 represented in FIG. 1, any other reset member such as, e.g., an air spring, an elastomer spring, a volute spring etc. capable of returning the plunger 3 to its original position may be braced in or on the handle 6 in another embodiment.

FIG. 6 shows a perspective overall view of an alternative embodiment of the biopsy needle. In such an alternative embodiment 100 a window 140 is integrated on the force transmitting element 8 in the guide sleeve. As the force transmitting element, which may be a PTFE tube by way of example, is nearly transparent while a synovial fluid or other body fluid is not, it is thus possible to realize quickly and easily whether the person carrying out the treatment is manipulating the biopsy needle 100 in the correct location.

FIGS. 6 and 7 further show that the trocar tip may also be provided without bores 22 in an alternative embodiment. The cutting blades as represented in FIG. 7 may furthermore by rounded, and the tip may have a hemispherical shape. As a result the person carrying out the treatment may be conferred better sensitivity concerning the actual location of the trocar tip in the tissue during a treatment. In addition this serves to avoid undesirable injury to the tissue.

The bores provided for fluid transfer and provided in the trocar tip in the description discussed in the foregoing may, in accordance with the representation in FIG. 6 and FIG. 8, alternatively (or additionally) be integrated in the cutting plunger in the form of transverse bores 130.

With a view to manufacturing technology and economy, the force transmitting piston may furthermore be realized not only in one part but also in two or more parts (170, 180). The individual parts may then simply be connected, e.g., by means of biocompatible adhesive, with locking, insertion, welded and/or soldered connections also being conceivable, of course. One embodiment of a composite realization is shown in FIG. 9.

In accordance with the representation in FIGS. 10 to 12, securing against rotation as discussed in the foregoing, which is achieved by means of the eccentric arrangement of the distal shaft of the force transmitting piston and the associated guide hole in a handle 200 and which may also serve for positional fixation, may alternatively also be established by means of a lever 150 urging a clamping member 160 via a thread 161 against a surface 181 of a part of the force transmitting piston. It is thus possible to obtain a clamping effect whereby the force transmitting piston 170, 180 may equally be held in a defined position. To this end a transverse bore 190 including a thread 191 as represented in FIG. 12 is provided in the handle. The clamping member may have such a construction that it may be screwed through the threaded bore 191 from inside the handle 200. This provides the advantage of also furnishing a stop in an upward direction. Subsequently the lever 150 may be attached by bonding or welding, etc. In order to facilitate the use of the present biopsy needle 100 for the person carrying out the treatment, the opening and locking direction of the lever may be indicated on the outside of the biopsy needle 100 in accordance with the representation in FIG. 6. Apart from this, the clamping mechanism need not be realized in accordance with the preceding description. The clamping member and the lever may, for example, also be realized of one unit. The threads 161, 191 may furthermore be replaced with an alternative force transmitting system.

As is represented in FIG. 13, the actuating device 9 may be mounted on the force transmitting piston so as to be freely rotatable. The actuating device 9 which is situated, e.g., on a distal journal 182 of the force transmitting piston 4, 170, 180, may be blocked by a counter disc 210 and the coupling means 10 which is adapted to be attached to the force transmitting piston 4, 170, 180 by means of a thread. Due to such a mount whereby the actuating device 9 may be rotated into a desired radial position and is thus rotatable relative to the handle 6 and/or the guide sleeve 11, the person carrying out the treatment is free to choose the hand for operating the joint biopsy needle. Moreover the joint biopsy needle is thus equally suitable for both left-handed and right-handed persons.

In alternative embodiments of the present biopsy needle it is possible to replace threads with smooth cylindrical surfaces, thus allowing to bond or weld connection parts; this furnishes the advantage of economic manufacture.

With a biopsy needle 100 in accordance with FIGS. 6 to 12 that is in the closed condition, i.e., having the cutting plunger 130 in the depressed condition, a person carrying out the treatment may penetrate into a body until a fluid becomes visible at the window 140. As soon as the fluid reaches the window 140, the person carrying out the treatment opens the biopsy needle 100 to thus release the pressure exerted on the cutting plunger 130, and attaches a syringe on a Luer connection 210 in order to generate a negative pressure. The subsequent manner of proceeding is the same as in the preceding description.

Although particular properties and features of the present biopsy needle are restricted to particular practical examples in the annexed figures, it should be self-evident that these may be employed in further embodiments presently not discussed.

The invention discussed in the foregoing thus provides a biopsy needle, in particular a joint biopsy needle comprising a trocar tip and a guide sleeve which adjoins a base surface of the trocar tip and which has a lateral opening in a region adjacent to the trocar tip. Moreover this biopsy needle has a plunger which can be displaced in the guide sleeve in the longitudinal direction thereof and has a cutting blade on a side that faces the trocar tip, wherein the tissue which is to be taken in the region of the lateral opening of the guide sleeve and can enter the guide sleeve through the lateral opening can be severed by the cutting blade. The guide sleeve is immobilized inside a handle. The guide sleeve is for the first time curved in at least one location between the trocar tip and the handle, wherein the plunger can be displaced by means of a flexible force transmitting element that is guided in the guide sleeve.

1.-20. (canceled)

21. A joint biopsy needle for taking tissue samples, comprising
a trocar tip,
a substantially rigid guide sleeve which adjoins a base surface of the trocar tip and which has a lateral opening adjacent to the trocar tip,
a plunger which can be displaced in the guide sleeve in the longitudinal direction thereof and has a cutting blade on a side that faces the trocar tip,
wherein the tissue which is to be taken into the lateral opening of the guide sleeve and can enter the guide sleeve through the lateral opening can be severed by the cutting blade, and
a handle in which the guide sleeve is immobilized,
wherein the guide sleeve is curved at least in one location between the trocar tip and the handle,
wherein the plunger can be displaced by a flexible force transmitting element that is guided in the guide sleeve,
wherein a force may be transmitted to the flexible force transmitting element via an actuating device and a force transmitting piston which is guided in the handle,
a distal shaft of the force transmitting piston and an associated guide hole in the handle are secured against rotation, and
the distal shaft of the force transmitting piston and the associated guide hole in the handle are eccentric and complementary relative to each other.

22. The joint biopsy needle according to claim 21, wherein the flexible force transmitting element is a plastic tube.

23. The joint biopsy needle according to claim 21, wherein the plunger has on its inside a hollow space which is open toward the cutting blade and in which the tissue to be collected may be received.

24. The joint biopsy needle according to claim 21, wherein the trocar tip is a cutting tip having a plurality of sides with concave cutting blades.

25. The joint biopsy needle according to claim 21, wherein on at least one side of the trocar tip there is at least one bore which allows a body fluid to flow through the trocar tip.

26. The joint biopsy needle according to claim 21, wherein on the distal end of the handle a coupling for a pressure generating device is formed and through which a drug may be injected into the region to be treated via a continuous cannulation through the handle, the force transmitting element, the plunger, and the trocar tip.

27. The joint biopsy needle according to claim 21, wherein a first reception is located at an end of the plunger facing the force transmitting piston, and a second reception for the force transmitting element is located at an end of the force transmitting piston facing the plunger.

28. The joint biopsy needle according to claim 21, wherein the force transmitting piston, whereby a force may be transmitted to the flexible force transmitting element, may be in the form of several parts and may be held in a defined position.

29. The joint biopsy needle according to claim 21, wherein the force transmitting piston may be held in a defined position a force which urges a clamping member against a surface of the force transmitting piston, the force being a lever which urges the clamping member against the surface of the force transmitting piston.

30. The joint biopsy needle according to claim 21, wherein a reset spring member for the plunger is arranged inside the handle.

31. The joint biopsy needle according to claim 21, wherein a window allowing direct viewing of the force transmitting element is integrated in the guide sleeve.

32. The joint biopsy needle according to claim 21, wherein the tip of the trocar tip is hemispherical.

33. The joint biopsy needle according to claim 21, wherein the cutting blades of the trocar tip are rounded.

34. The joint biopsy needle according to claim 21, wherein transverse bores are integrated in the plunger.

35. The joint biopsy needle according to claim 21, wherein the actuating device is arranged on the force transmitting piston so as to be freely rotatable about the longitudinal axis of the joint biopsy needle.

36. A method for taking tissue samples by means of a joint biopsy needle of claim 1, comprising the steps of:
a) introducing the joint biopsy needle into a region to be examined in a body,
b) once the joint biopsy needle has been introduced into a desired position, actuating the plunger via the actuating device, the force transmitting piston, and the force transmitting element so as to sever tissue to be collected with the cutting blade in the region of the lateral opening of the guide sleeve and which could enter the guide sleeve through the lateral opening and receive it in the hollow space of the plunger,
c) once the tissue to be taken is inside the hollow space, extracting the biopsy needle from the region to be examined,
d) after the joint biopsy needle was extracted from the body, removing the tissue sample from the hollow space.
